(19) United States
(12) Patent Application Publication
(10) Pub. No.: US 2024/0261567 A1
(43) Pub. Date: Aug. 8, 2024

(54) SHIFTABLE AND FLEXIBLE TRANSDUCER ARRAYS WITH LAYER OF ANISOTROPIC MATERIAL

(71) Applicant: Novocure GmbH, Root D4 (CH)

(72) Inventors: Stas OBUCHOVSKY, Haifa (IL); Yoram WASSERMAN, Haifa (IL); David SHAPIRO, Haifa (IL)

(73) Assignee: Novocure GmbH, Root D4 (CH)

(21) Appl. No.: 18/434,777

(22) Filed: Feb. 6, 2024

Related U.S. Application Data
(63) Continuation-in-part of application No. 18/432,933, filed on Feb. 5, 2024.
(60) Provisional application No. 63/443,585, filed on Feb. 6, 2023.
(60) Provisional application No. 63/523,491, filed on Jun. 27, 2023.

(51) Int. Cl. A61N 1/04; A61N 1/36

(52) U.S. Cl. CPC A61N 1/0476; A61N 1/0496; A61N 1/36002

(57) ABSTRACT

A transducer apparatus for delivering tumor treating fields to a subject's body includes: an array of electrodes, the array configured to be positioned over the subject's body with a face of the array facing the subject's body, the array comprising electrode elements positioned in substantially symmetrical positions arranged around a centroid of the array; a void space located between at least one pair of adjacent electrodes of the array; and a polymeric material layer overlaying the array of electrodes and located on a side of the array facing away from the subject's body.

### CROSS-REFERENCE TO RELATED APPLICATIONS

This Application claims priority to U.S. Provisional Application No. 63/443,585, filed Feb. 6, 2023, U.S. Provisional Application No. 63/523,491, filed Jun. 27, 2023, and U.S. patent application Ser. No. 18/432,933, filed Feb. 5, 2024, the contents of each of which are incorporated by reference herein in their entirety.

### BACKGROUND

Tumor treating fields (TTFields) are low intensity alternating electric fields within the intermediate frequency range (for example, 50 kHz to 1 MHz), which may be used to treat tumors as described in U.S. Pat. No. 7,565,205. TTFields are induced non-invasively into the region of interest by transducers placed on the patient's body and applying AC voltages between the transducers. Conventionally, a first pair of transducers and a second pair of transducers are placed on the subject's body. AC voltage is applied between the first pair of transducers for a first interval of time to generate an electric field with field lines generally running in the front-back direction. Then, AC voltage is applied at the same frequency between the second pair of transducers for a second interval of time to generate an electric field with field lines generally running in the right-left direction. The system then repeats this two-step sequence throughout the treatment.

### BRIEF DESCRIPTION OF THE DRAWINGS

FIG. 1 depicts an example of transducers located on a subject's head.

FIG. 2 depicts an example of transducers located on a subject's body.

FIGS. 3A-3D are cross-sectional views of example structures of transducers.

FIGS. 3E and 3F are top and cross-sectional views, respectively, of another example structure of a transducer.

FIGS. 4A and 4B depict an example layout of an array of electrode elements on a transducer apparatus (FIG. 4A) and a similar array comprising cuts or slits in the bandage overlay (FIG. 4B).

FIGS. 4C and 4D depict an example layout of an array of electrode elements on a transducer apparatus (FIG. 4C) and the array after stretching in opposing directions (FIG. 4D).

FIG. 4E depicts an example layout of an array of electrode elements on a transducer apparatus.

FIG. 4F depicts an example layout of an array of electrode elements on a transducer apparatus.

FIG. 4G depicts an example layout of an array of electrode elements on a transducer apparatus.

FIGS. 5A, 5B, and 5C depict an example layout of an array of electrode elements on a transducer apparatus wherein the array is paired with a bandage overlay.

FIG. 6 is an example method of applying TTFields to a subject's body in accordance with the present techniques.

### DESCRIPTION OF EMBODIMENTS

This application describes exemplary transducer apparatuses used to apply TTFields to a subject's body for treating one or more cancers.

Transducers used to apply TTFields to a subject's body often include multiple electrode elements electrically coupled together on a substrate and attached to the subject's body at a desired location, for example, via an adhesive backing of the substrate or a separately applied adhesive. Conventional transducers have large, rectangular surfaces so as to maximize a number of electrode elements that are located on the transducer for applying TTFields to the subject's body. However, subjects can experience skin irritation on portions of their skin that are contacted by the electrode elements during TTField treatment. Such irritation may be more common at positions directly underneath the electrode elements, where heat and current may be at their highest concentrations, particularly for electrodes around the outer edge of the array.

The inventors have now recognized that a need exists for transducers that can reduce, minimize, prevent, soothe, heal, or treat skin irritation without significantly changing the field intensity of TTFields being induced in the subject's body. For example, transducers that are able to be shifted so that skin previously contacted by electrode elements can be uncovered (or covered by a topical medication) without substantially moving the transducer from an optimal location on the subject's body are desired. The new position of the transducer after shifting is in substantially the same location if the footprint of the new position after shifting covers greater than or equal to 80% of the footprint of the original position before shifting; or if it covers greater than or equal to 90% of the footprint of the original position before shifting; or if it covers greater than or equal to 95% of the footprint of the original position before shifting. In some embodiments, the footprint of the new position of the transducer after shifting covers 100% of the footprint of the original position of the transducer before shifting. The shifting of the transducer apparatuses can reduce, minimize, prevent, soothe, heal, and/or treat skin irritation while maintaining the transducer in an optimal location on the subject's body. As a result, the transducers can continuously induce TTFields at an ideal location and power level for targeting a region of interest (e.g., tumor) in the subject's body, thereby improving patient outcomes.

In some embodiments, the disclosed transducer apparatuses can be shifted via rotation about a centroid of the array of electrodes, or via translation of the array of electrodes, so that one or more portions of the subject's skin that were previously contacted by electrode elements can be uncovered (or covered by a medication), while maintaining an optimal location of the transducer on the subject's body. In some embodiments, the array of electrodes does not include an electrode position that encompasses the centroid of the array. The disclosed transducer apparatuses may have a substantially rounded shape enabling the transducers to be positioned on a subject's head. In other examples, the disclosed transducer apparatus may have other (e.g., non-rounded) shapes.

The disclosed transducer apparatuses may also include a layer of anisotropic material located on a side of the array of electrode elements facing the subject's body. Such a layer of anisotropic material may spread the heat and/or current generated at the individual electrode elements within a plane that is perpendicular to the direction from the electrode elements to the subject's body. Spreading heat and/or current in this plane may reduce the concentration of heat and/or current at locations directly under the individual electrode elements, thus reducing the amount or severity of irritation, if any, that occurs on the subject's skin. The transducer apparatus having a layer of anisotropic material as described herein may also be shiftable (e.g., via rotation or translation) to further reduce, minimize, prevent, soothe, heal, and/or treat skin irritation.

Use of a layer of anisotropic material as disclosed herein may spread heat and/or current over a greater area of the subject's skin thereby reducing skin irritation compared to transducer arrays without a layer of anisotropic material. However, covering a greater area, for example, on the torso, can make everyday movements of the body feel restrictive because the transducer apparatus with the layer of anisotropic material is largely inflexible and cannot stretch with movements of the body. Moreover, in the extremes of body movement, the adhesive may prove to be aggressive and damage the skin, or the adhesive may prove to be unsuccessful in securing the array in place and may come loose. What is needed is a more flexible transducer array, wherein the flexibility can help absorb the stress forces between the adhesive and the area of the subject's skin that is covered by the array when body movement causes such stress forces.

The disclosed transducer apparatuses may be configured to stretch in one or more directions. The apparatuses may be capable of stretching such that a void space located between at least one pair of adjacent electrodes of the array is increased in size due to movement of one or more electrodes of at least one pair of adjacent electrodes of the array in a direction away from one another. The apparatuses may be stretched upon, or after, application to a subject to improve adherence to the subject.

The disclosed transducer apparatuses may also include a flexible bandage layer. The flexible bandage layer, for example a polymeric material layer (e.g., a polyurethane layer), secures the array of electrodes to a subject while also providing a degree of protection. The polyurethane layer may take the form of a polyurethane polymer film or bandage (or polyurethane dressing), and is stretchable in the plane of the film in multiple directions. Accordingly, the flexible bandage layer also improves the flexibility of the disclosed transducer apparatuses.

Descriptions of embodiments related to specific exemplary Figures herein may be applicable, and may be combined with, descriptions of embodiments related to other exemplary Figures herein unless otherwise indicated herein or otherwise clearly contradicted by context.

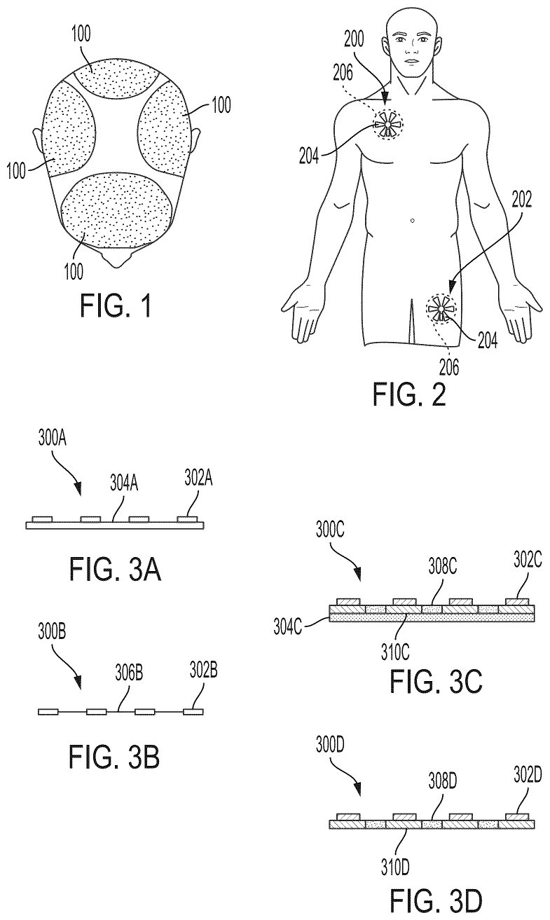

FIG. 1 depicts transducers 100 positioned on the head of a subject's body. Such arrangement of transducers 100 is capable of applying TTFields to a tumor in a region of the subject's brain. Various other positions and/or orientations on the subject's head may be selected for placement of transducers. Each transducer 100 may have an array of electrode elements disposed thereon. Each transducer 100 may be placed on a subject's head with a face of the array of electrode elements facing and conforming to the subject's head. As illustrated, the transducers 100 on the subject's head do not overlap one another, e.g., due to their rounded shape.

FIG. 2 depicts transducers 200 and 202 attached to other portions (e.g., a thorax/torso and a thigh) of the subject's body. The transducers 200 and 202 may be affixed to the subject's body via a medically appropriate gel or adhesive. In other embodiments, the transducers 200 and 202 may be attached to one or more garments and held against the subject's body. Each of the transducers 200 and 202 may have an array of electrode elements 204 disposed thereon. Each transducer 200 and 202 may be placed over the subject's body with a face of the array of electrode elements facing and conforming to the subject's body.

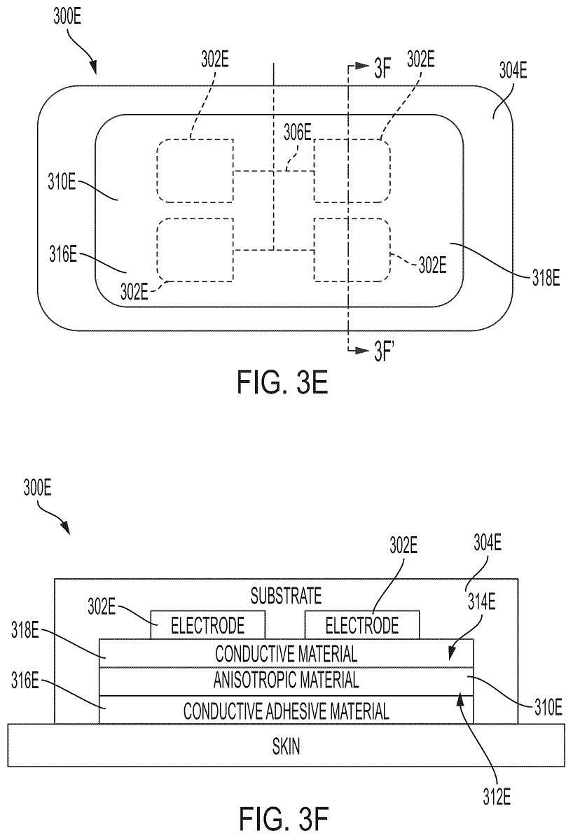

In the first transducer 200 and the second transducer 202, an outer perimeter 206 (defined by a dashed line in FIG. 2) traces the array of electrode elements 204. In an example, the outer perimeter 206 of the array on each transducer may have a substantially rounded edge. The outer perimeter 206 (or outer perimeter for any array herein) may be substantially circular, oval, ovaloid, ovoid, or elliptical in shape. For example, as illustrated, the outer perimeter 206 may have a circular shape. In another example, the outer perimeter 206 (or outer perimeter for any array herein) may have other shapes such as, for example, a square or rectangular shape or substantially square or rectangular shape with rounded corners (e.g., as shown in FIG. 3E).

The structure of the transducers may take many forms. In FIG. 3A, the transducer 300A has a plurality of electrode elements 302A positioned on a substrate 304A. The substrate 304A is configured for attaching the transducer 300A to a subject's body. Suitable materials for the substrate 304A include, for example, cloth, foam, flexible plastic, and/or a conductive medical gel. The transducer 300A may be affixed to the subject's body via the substrate 304A (e.g., via an adhesive layer and/or a conductive medical gel). The adhesive layer that contacts the subject's skin may be present around the outer perimeter of the array of electrodes, and/or may be present between one or more gaps between electrodes. Alternatively, areas between electrodes may be non-adhesive regions. The transducer may be conductive or non-conductive. FIG. 3B depicts another example of the structure of the transducer 300B. In this example, the transducer 300B includes a plurality of electrode elements 302B that are electrically and mechanically connected to one another without a substrate. In one example, electrode elements 302B are connected to each other through conductive wires 306B.

In FIGS. 3C and 3D, the transducers 300C and 300D include one or more medication regions 308C and 308D, respectively. The medication regions 308C and 308D may be non-adhesive regions. For example, no exposed adhesive is present in the medication region(s) 308C and 308D. The medication region(s) 308C and 308D may each include a medication substrate. The medication substrate may be capable of at least one of receiving, absorbing, or holding a topical medication applied thereto. The medication substrate may include a cloth, a gauze, a non- woven material, a foam, or a sponge located between one or more pairs of electrode elements 302C or 302D. In an example, the medication region(s) 308C and 308D may also include a topical medication integrated in or on the medication substrate. The topical medication may include a base component of oil, water, petrolatum, wax, cellulose, or a combination thereof. The topical medication may be a cream, an ointment, a lotion, a gel, a wax, a paste, or a mineral oil jelly. The topical medication may include at least one of an antibiotic, a steroid, an antiseptic, an emollient, an anesthetic, a terpene, a plant extract, a silicon-based organic polymer, an antifungal agent, a burn relief agent, a skin repair agent, an astringent, or an antihistamine. The topical medication may be any desired compound capable of soothing, healing, and/or providing relief for inflammation, sores, or other irritation that may develop on the skin of the subject's body. The topical medication may be substantially evenly distributed through a thickness of the medication substrate to form the medication regions 308C and 308D. Alternatively, the topical medication may be substantially disposed on the surface of the medication substrate to form the medication regions 308C and 308D.

As shown in FIG. 3C, the transducer 300C may include a transducer substrate 304C that is separate from the medication region(s) 308C. The array of electrode elements 302C may be disposed on a surface of the transducer substrate 304C, and the transducer substrate 304C may include an adhesive layer 310C for attaching the transducer apparatus to the subject's body. The medication substrate may be a portion of the transducer substrate 304C, or may be disposed on the surface of the transducer substrate 304C. Thus, the medication region 308C may be disposed on the surface of the transducer substrate 304C (as shown in FIG. 3C). In other embodiments, for example as shown in FIG. 3D, the transducer 300D may not include a transducer substrate, but rather merely an adhesive layer 310D for attaching the transducer 300D to the subject's body, and the medication region(s) 308D may be coupled between different portions of the adhesive layer 310D and span a distance between the electrode elements 302D.

FIGS. 3E and 3F depict another example transducer 300E. FIG. 3F is a cross-sectional view of the transducer 300E shown in FIG. 3E, as viewed at 3F-3F′. The transducer 300E includes a plurality of electrode elements 302E positioned on a substrate 304E, similar to the substrate 304A described above with reference to FIG. 3A. The substrate 304E is configured for attaching the transducer 300E to a subject's body. The electrode elements 302E may be connected to each other through conductive wires 306E.

The transducers 300A, 300B, 300C, 300D, and 300E may include arrays of substantially flat electrode elements 302A, 302B, 302C, 302D, and 302E, respectively. The array of electrode elements may be capacitively coupled. The electrode elements 302A, 302B, 302C, 302D, and 302E may be non-ceramic dielectric materials positioned over a plurality of flat conductors such as, for example, polymer films disposed over pads on a printed circuit board or over flat pieces of metal. In another example, the electrode elements 302A, 302B, 302C, 302D, and 302E are ceramic elements. In another example, the electrode elements do not have a dielectric material.

In some embodiments, the dielectric material of the electrode elements 302A, 302B, 302C, 302D, and 302E can have a dielectric constant ranging from 10 to 50,000. In some embodiments, the layer of dielectric material includes a high dielectric polymer material such as poly(vinylidene fluoride-trifluoroethylene-chlorotrifluoroethylene) and/or poly(vinylidene fluoride-trifluoroethylene-1-chlorofluoroethylene). Those two polymers are abbreviated herein as “Poly(VDF-TrFE-CTFE)” and “Poly(VDF-TrFE-CFE),” respectively. The dielectric constant of these materials is on the order of 40. In some embodiments, the polymer layer can be poly(vinylidene fluoride-trifluoroethylene-chlorotrifluoroethylene-chlorofluoroethylene) or “Poly(VDF-TrFE-CTFE-CFE).”

In some embodiments, the layer of dielectric material of the electrode elements 302A, 302B, 302C, 302D, and 302E includes a terpolymer comprising polymerized units of monomers such as VDF, TrFE, CFE and/or CTFE in any suitable molar ratio. Suitable terpolymers include those, for example, having 30 to 80 mol % VDF, 5 to 60 mol % TrFE, with CFE and/or CTFE constituting the balance of the mol % of the terpolymer.

On transducer arrays that include multiple electrode elements, the portions of the transducer arrays positioned directly beneath the electrode elements may become hotter than the portions of the transducer arrays positioned between the electrode elements. Furthermore, higher currents flow through the electrode elements that may be located along the edge of the array compared to the electrode elements located toward the middle of the array. Further still, an electrode element located at a corner or similar sharp bend in the edge of the array may have a higher current than other electrode elements along the edge and near the center of the array.

An uneven distribution of current through the transducer array can lead to higher temperature zones (or “hot spots”) e.g., at the corners or edges of the transducer array, which, in turn, may limit the maximum operational current that may be driven by a transducer array and, as a result, the strength of the resulting TTFields.

Optionally, as shown in FIGS. 3E and 3F, embodiments described herein may incorporate into the transducer (e.g., 300E in FIGS. 3E and 3F) a layer of anisotropic material (e.g., 310E in FIGS. 3E and 3F). As shown, the layer of anisotropic material 310E has a front face 312E and a back face 314E, wherein the back face 314E faces the array of electrode elements 302E. The layer of anisotropic material 310E has anisotropic thermal properties and/or anisotropic electrical properties. If the layer of anisotropic material 310E has anisotropic thermal properties (for example, greater thermal conductivity in the plane of the layer than through the plane of the layer), then the layer spreads the heat out more evenly over a larger surface area. If the layer of anisotropic material 310E has anisotropic electrical properties (for example, greater electrical conductivity in the plane of the layer than through the plane of the layer), then the layer spreads the current out more evenly over a larger surface area. In each case, this lowers the temperature of the hot spots and raises the temperature of the cooler regions when a given AC voltage is applied to the array of electrode elements. Accordingly, the current can be increased (thereby increasing the therapeutic effect) without exceeding the safety temperature threshold at any point on the subject's skin.

In some embodiments, the layer of anisotropic material 310E is anisotropic with respect to electrical conductivity properties. In some embodiments, the layer of anisotropic material 310E is anisotropic with respect to thermal conductivity properties. In some preferred embodiments, the layer of anisotropic material 310E is anisotropic with respect to both electrical conductivity properties and thermal conductivity properties.

The anisotropic thermal properties include directional thermal properties. Specifically, the layer of anisotropic material 310E may have a first thermal conductivity in a direction that is perpendicular to its front face (skin-facing surface) 312E that is different from a thermal conductivity of the layer of anisotropic material 310E in directions that are parallel to the front face 312E. For example, the thermal conductivity of the layer of anisotropic material 310E in directions parallel to the front face 312E is more than two times higher than the first thermal conductivity. In some preferred embodiments, the thermal conductivity in the parallel directions is more than ten times higher than the first thermal conductivity. For example, the thermal conductivity of the sheet in directions that are parallel to the front face 312E may be more than: 1.5 times, 2 times, 3 times, 5 times, 10 times, 20 times, 100 times, 200 times, or even more than 1,000 times higher than the first thermal conductivity.

The anisotropic electrical properties include directional electrical properties. Specifically, the layer of anisotropic material 310E may have a first electrical conductivity (or, conversely, resistance) in a direction that is perpendicular to its front face 312E that is different from an electrical conductivity (or resistance) of the layer of anisotropic material 310E in directions that are parallel to the front face 312E. For example, the resistance of the layer of anisotropic material 310E in directions parallel to the front face 312E may be less than the first resistance. In some preferred embodiments, the resistance in the parallel directions is less than half of the first resistance or less than 10% of the first resistance. For example, the resistance of the layer of anisotropic material 310E in directions that are parallel to the front face 312E may be less than: 75%, 50%, 40%, 30%, 20%, 10%, 5%, 1%, 0.5%, or even less than 0.1% of the first resistance.

In some embodiments (e.g., when the layer of anisotropic material 310E is a sheet of pyrolytic graphite), the layer of anisotropic material 310E has both anisotropic electrical properties and anisotropic thermal properties.

The layer of anisotropic material 310E may include graphite (e.g., a sheet of graphite). Examples of suitable forms of graphite include synthetic graphite, such as pyrolytic graphite (including, but not limited to, Pyrolytic Graphite Sheet (PGS), available from Panasonic Industry, Kadoma, Osaka, Japan), other forms of synthetic graphite, including but not limited to, graphite foil made from compressed high purity exfoliated mineral graphite (including, but not limited to, that supplied as MinGraph® 2010A Flexible Graphite, available from Mineral Seal Corp., Tucson, Arizona, USA), or graphitized polymer film, e.g., graphitized polyimide film, (including, but not limited to, that supplied by Kaneka Corp., Moka, Tochigi, Japan). In alternative embodiments, conductive anisotropic materials other than graphite may be used instead of graphite.

In some embodiments, the layer of anisotropic material 310E is a sheet of pyrolytic graphite. Thermal conductivity of pyrolytic graphite sheets in directions that are parallel to the front face 312E of those sheets is typically more than 50 times higher than the thermal conductivity of those sheets in directions that are perpendicular to the front face 312E. And electrical resistivity of pyrolytic graphite sheets in directions that are parallel to the front face 312E of those sheets is typically less than 2% of the electrical resistivity of those sheets in directions that are perpendicular to the front face 312E.

The transducer 300E may further include at least one layer of conductive adhesive material 316E disposed on a front facing side of the layer of anisotropic material 310E. In some embodiments, the at least one layer of conductive adhesive material 316E may be disposed on the front face 312E of the layer of anisotropic material 310E. The at least one layer of conductive adhesive material 316E may have a biocompatible front surface. Note that in the embodiment illustrated in FIG. 3F, there is only a single layer of conductive adhesive material 316E, and that single layer (the front layer) is biocompatible. But in alternative embodiments, there could be more than one layer, in which case only the front layer may be biocompatible, or the front layer and one or more other layers may be biocompatible. In the FIG. 3F embodiment, the front layer of conductive adhesive material 316E is configured to ensure good electrical contact between the device and the body. In some embodiments, the front layer of conductive adhesive material 316E should cover the entire front face 312E of the layer of anisotropic material 310E. The front layer of conductive adhesive material 316E may be the same size (area) or larger than the layer of anisotropic material 310E. In some embodiments, the front layer of conductive adhesive material 316E includes hydrogel. In these embodiments, the hydrogel may have a thickness between 50 and 2,000 μm. In other embodiments, the front layer of conductive adhesive material 316E includes a conductive adhesive composite as further disclosed herein.

The transducer 300E may further include a first layer of conductive material 318E positioned between the array of electrode elements 302E and the back face 314E of the layer of anisotropic material 310E facing the array. The first layer of conductive material 318E facilitates the electrical contact between the array of electrode elements 302E and the back face 314E of the layer of anisotropic material 310E. In some embodiments, the layer of conductive material 318E is a layer of hydrogel. In other embodiments, a different conductive material (e.g., conductive grease, conductive adhesives, conductive tape, etc.) could be used. For example, the layer of conductive material 318E may include a conductive adhesive composite as further disclosed herein.

In some embodiments, the at least one layer of conductive adhesive material 316E and/or the layer of conductive material 318E is a single layer of non-hydrogel conductive adhesive such as the developmental product FLX068983—FLEXcon® OMNI-WAVE™ TT 200 BLACK H-502 150 POLY H-9 44PP-8 from FLEXcon, Spencer, MA, USA, or other such OMNI-WAVE products from FLEXcon; or ARcare® 8006 electrically conductive adhesive composition manufactured and sold by Adhesives Research, Inc. (Glen Rock, PA, USA). Non-hydrogel conductive adhesives may include a waterless polymer with adhesive properties and carbon particles, powder, fibers, flakes, granules and/or nanotubes. The adhesive polymer may be, for example, an acrylic polymer or a silicone polymer, or combination thereof, which may be available as acrylic-or silicone-based carbon-filled adhesive tapes. The adhesive may additionally include one or more conductive polymers (such as, for example, polyaniline (PANI), or poly(3,4-ethylenedioxythiophene) (PEDOT), or others known in the art). The conductive filler in the at least one layer of conductive adhesive material 316E or conductive material 318E may be non-metallic. In these embodiments, the conductive adhesive may have a thickness between 10 and 2,000 μm, such as, from 20 to 1,000 μm, or 30 to 400 μm.

In some embodiments, the transducer 300E may be constructed using a pre-formed 3-(or more) layer laminate comprising the conductive material 318E, the layer of anisotropic material 310E, and the at least one layer of conductive adhesive material 316E, wherein the at least one conductive adhesive material 316E and the conductive material 318E are both conductive adhesive composites as described above, and the layer of anisotropic material 310E is a thin sheet of synthetic graphite such as pyrolytic graphite, as described above. The at least one conductive adhesive material 316E and the conductive material 318E may be the same material or may be different. By way of example, in an embodiment, both the conductive adhesive material 316E and the conductive material 318E may include an acrylic polymer and a carbon powder filler; or both the conductive adhesive material 316E and the conductive material 318E may include an acrylic polymer and a carbon fiber filler. In another embodiment, the conductive adhesive material 316E includes an acrylic polymer and a carbon fiber filler, and the conductive material 318E include an acrylic polymer and a carbon powder filler; or vice-versa.

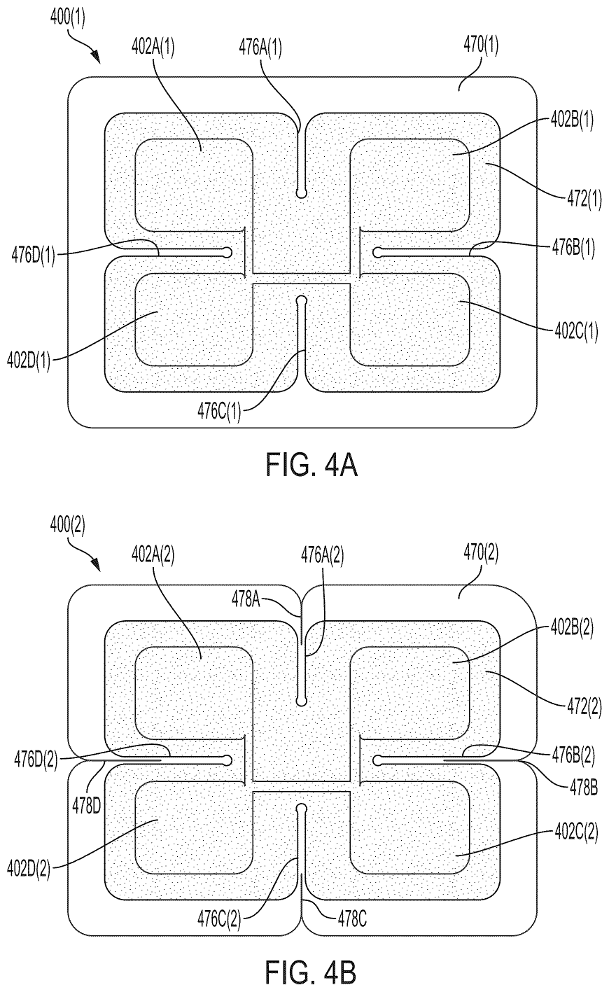

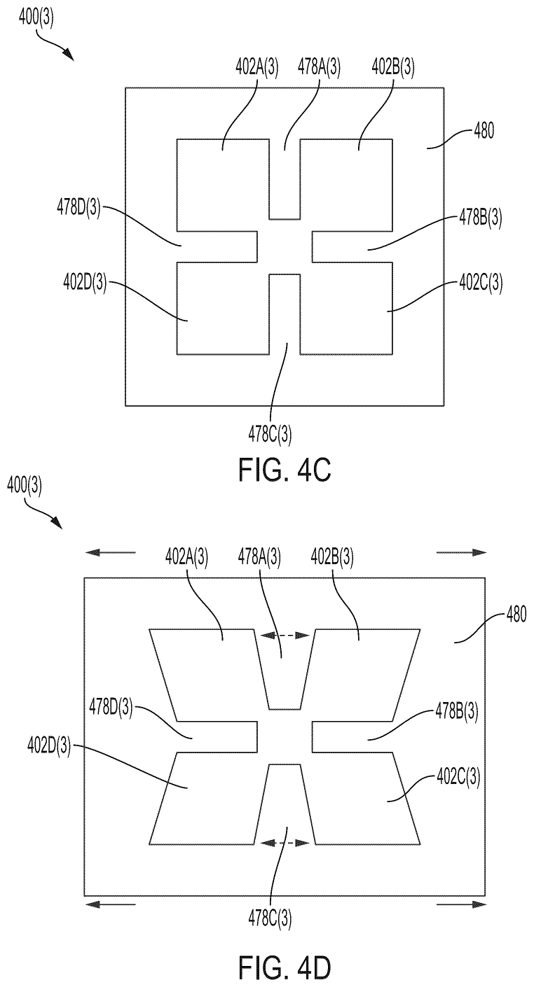

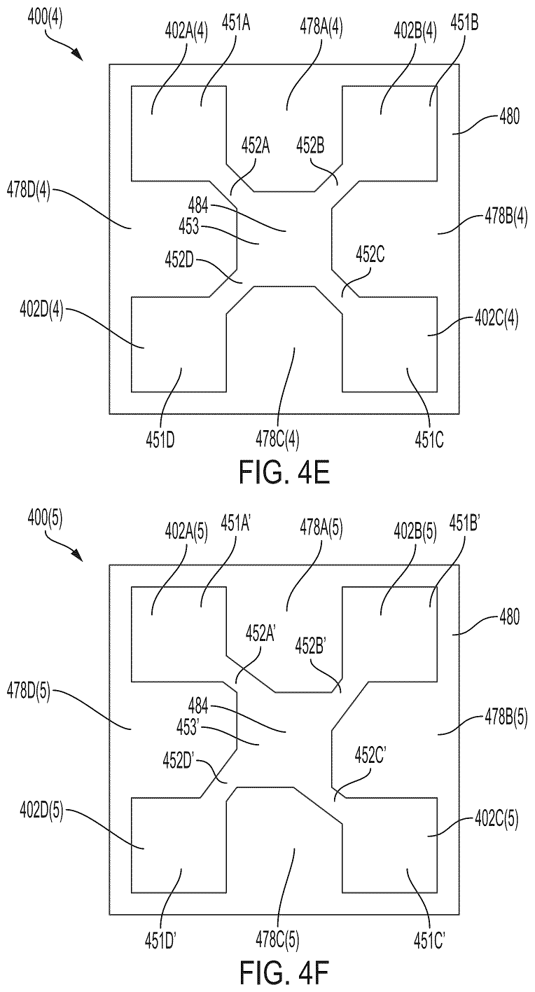

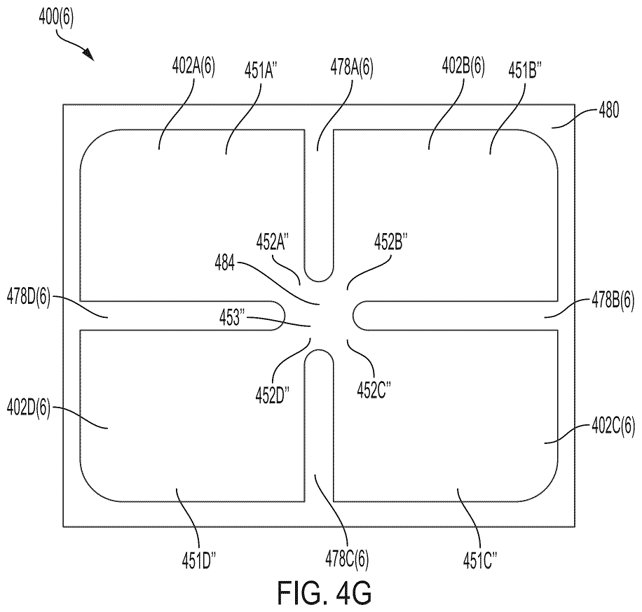

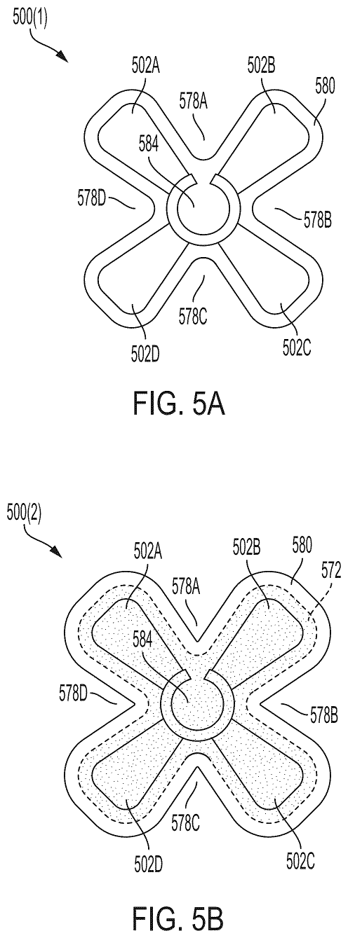

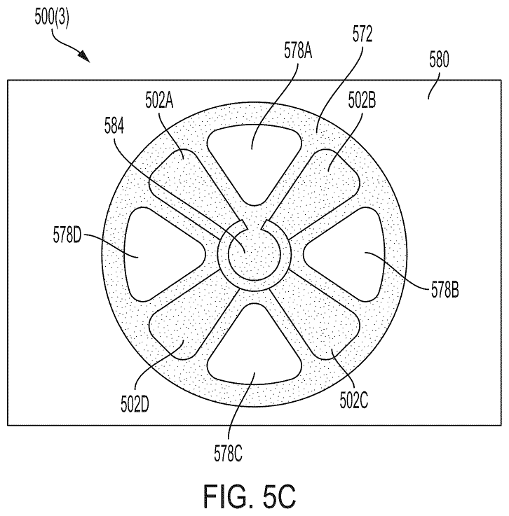

FIGS. 4A-5C illustrate further examples of transducer apparatuses that may be used to apply TTFields to a subject's body. The transducer apparatuses of FIGS. 4A-4C and 4E-5C include at least four electrodes arranged around a centroid and having point symmetry (for example, ignoring any central connecting bridge between electrodes, the arrays in FIGS. 4A, 4B and 4G have C2 symmetry; the arrays in FIGS. 4C, 4E, 4F, 5A, 5B and 5C have C4 symmetry).

FIGS. 4A and 4B depict example transducer apparatuses 400(1) and 400(2). The transducer apparatuses 400(1) and 400(2) each include a substrate layer (470(1), 470(2)) and an array of electrodes (402A(1)-402D(1), 402A(2)-402D(2)) (i.e., 402(1), 402(2) disposed on the substrate layer (470(1), 470(2)). In some embodiments, the substrate layer 470 may be a bandage overlay, wherein at least the outer perimeter may be coated with an adhesive layer on the skin-facing side in order to secure the transducer array to the subject's skin. The array is configured to be positioned over the subject's body with a face of the array facing the subject's body. The transducer apparatuses 400(1) and 400(2) also include a layer of anisotropic material (472(1), 472(2)) electrically coupled to the array of electrodes (402(1), 402(2)) and located on a side of the array opposite the substrate (or bandage overlay) layer (470(1), 470(2)). The layer of anisotropic material (472(1), 472(2)) may have at least one cut or slit (476A(1)-476D(1), 476A(2)-476D(2)) (i.e., 476(1), 476(2)) formed through a full thickness of the layer of anisotropic material (472(1), 472(2)). As shown, each cut or slit (476(1), 476(2)) may extend from an outer edge of the layer of anisotropic material (472(1), 472(2)) toward a center portion of the layer of anisotropic material (472(1), 472(2)) when viewed in a direction perpendicular to the face of the array. The cut(s) or slit(s) (476(1), 476(2)) allow the layer of anisotropic material (472(1), 472(2)) to separate enough (at the slit regions) to provide some flexibility for stretching, twisting, or other movement of the subject's body when the transducer apparatus (400(1), 400(2)) is attached to the subject's body. In the transducer apparatus 400(1) of FIG. 4A, the substrate layer 470(1) does not include any cuts or slits. In the transducer apparatus 400(2) of FIG. 4B, the substrate layer 470(2) has at least one cut or slit 478A-478D (i.e., 478) formed through a full thickness of the substrate layer 470(2), the cut or slit 478 extending from an outer edge of the substrate layer 470(2) toward a center portion of the substrate layer 470(2) when viewed in the direction perpendicular to the face of the array. As illustrated, the cuts or slits 478 formed in the substrate layer 470(2) may be at least partially coincident with the cuts or slits 476(2) formed in the layer of anisotropic material 472(2). The transducer apparatuses 400(1) and 400(2) of FIGS. 4A and 4B have increased flexibility compared to transducers that do not feature such cuts or slits formed in the layer of anisotropic material and/or substrate layer. The cuts or slits may be applied to transducers having any desired shape, number, and arrangement of electrodes, not just those configured to provide relief areas in response to stretching (e.g., FIGS. 4C-5C).

The substrate layers 470(1) and 470(2) in FIGS. 4A and 4B, respectively, can be a bandage overlay which may be a polymeric material layer, such as, for example, a flexible polyurethane film or bandage. In some embodiments, the bandage overlay is a clear transparent flexible polyurethane polymer film or bandage. Preferably, the flexible polyurethane film or bandage can stretch in multiple directions in the plane of the film. The flexible polyurethane film or bandage may work in concert with the slits or cuts in the layer of anisotropic material to provide flexibility for the transducer array. The same type of flexible bandage overlay (such as, for example, the polyurethane polymer film or bandage, described in more detail below) may be used in conjunction with other embodiments described herein. For all of the embodiments disclosed herein, an additional bandage overlay, such as a Coban™ bandage (3M, St. Paul, MN, USA), or other self-adherent wrap, may be employed in addition to, or in place of, the polyurethane polymer film or bandage.

In FIGS. 4C-4G and 5A, for the sake of clarity, the electrode positions and layer of anisotropic material are not shown separately. Each of the figures illustrates a central 4-lobed trace. In some embodiments, the central 4-lobed trace represents the areal footprint of the electrodes and there is no layer of anisotropic material present. In some embodiments, the central 4-lobed trace may represent coincident areal footprints of both the electrode positions and the layer of anisotropic material (and may also include one or more coincident areal traces of conductive adhesive regions as well). In some embodiments, the central 4-lobed trace may represent the areal footprint of the layer of anisotropic material, with areal traces of the electrode positions obscured but with a smaller areal footprint than that of the layer of anisotropic material, for example, as shown in FIGS. 4A and 4B.

In some embodiments, the transducer apparatus enables a simple rotation of the transducer to reposition at least one void region (which may be a non-adhesive void region formed in the electrode array, or, alternatively, at least one medication region as described above with reference to FIGS. 3C and 3D) over an area of the subject's skin that was previously covered by an electrode element. Positioning a void region over the area of the subject's skin that was previously covered by an electrode element allows this area of the subject's skin to “breathe” and recover from the prior contact it had with the electrode element used to induce TTFields. The relative positioning of electrode elements and void regions (or medication regions) disclosed herein may be used along with the layer of anisotropic material (e.g., 310E of FIGS. 3E and 3F) described above to further reduce irritation of the subject's skin.

As some subjects experience skin irritation in response to prolonged interaction of the skin with the electrode elements used to induce TTFields, moving the transducer so that a void is positioned over an affected area of the subject's skin may help to minimize, reduce, or prevent irritation of the subject's skin throughout TTField treatment. In addition, positioning a medication region over the area of the subject's skin that was previously covered by an electrode element allows an application of a topical medication to this area of the subject's skin to soothe, heal, reduce inflammation or soreness, or otherwise improve the condition of the subject's skin. In addition, spreading heat and/or current in a plane perpendicular to the direction from the electrode elements to the subject's skin may allow for a reduction in the heat and/or current at any particular location above the subject's skin, thereby reducing overall skin irritation. Since the transducer apparatus may be rotated about a centroid of the array of electrodes, this allows the transducer to continue outputting TTFields from the same optimal location on the subject's body during treatment while providing relief and/or healing to areas of the subject's skin.

FIGS. 4C and 4D depict other example transducer apparatuses 400(3) for which similar features and labelling notations are present as those described with respect to embodiments of FIGS. 4A and 4B. The transducer apparatus 400(3) includes a bandage overlay 480. In some embodiments, the bandage overlay 480 is composed of polyurethane. In some embodiments, the bandage overlay 480 may be a polyurethane polymer film or bandage, as above (470) and elsewhere herein. In some embodiments, the bandage overlay 480 (and 470 and elsewhere herein) has a thickness of less than or equal to 250 μm, or less than or equal to 200 μm, or less than or equal to 160 μm, such as, for example, in a range of from 50-250 μm, or from 50-200 μm, or from 50-160 μm. In further embodiments, the bandage overlay 480 has a thickness in a range of from 80-160 μm, or from 100-140 μm. In some embodiments, the bandage overlay 480 is disposed over the array of electrodes 402A(3)-402D(3) such that the bandage overlay 480 covers the array of electrodes 402A(3)-402D(3) and void spaces 478A(3)-478D(3) between the electrodes 402A(3)-402D(3). The transducer apparatus 400(3) may be capable of stretching such that a void space 478 located between at least one pair of adjacent electrodes 402 of the array is increased in size due to movement of one or more electrodes 402 of at least one pair of adjacent electrodes 402 of the array in a direction away from one another, for example as shown in FIG. 4D. In some embodiments, the transducer apparatus 400(3) may be configured to stretch upon or after application of the apparatus on a subject. The size and shape of the electrodes are not restricted to those shown in the Figures. A similar bandage overlay may be used in conjunction with other embodiments described herein.

FIGS. 4E, 4F, and 4G depict other example transducer apparatuses 400(4), 400(5), and 400(6). The transducer apparatuses 400(4), 400(5), and 400(6) each include a bandage overlay 480 as described above with regards to apparatus 400(3). In discussing the electrode positions, to be concise, the illustrated areal trace is assumed to be representative of the shapes of the electrodes (that is, either the electrodes alone without any layer of anisotropic material; or representative of both the areas of the electrodes and of the area of the layer of anisotropic material, the areas being coincident). However, as discussed above, alternative embodiments exist for which the illustrated areal trace represents the areal trace of the layer of anisotropic material, and the areal trace of the electrodes is a subset of the illustrated areal trace. The transducer apparatuses 400(4), 400(5), and 400(6) demonstrate different shapes of the array of electrodes 402A(4)-402D(4), 402A(5)-402D(5), and 402A(6)-402D(6). In FIG. 4E, each electrode 402A(4)-402D(4) includes a lobe 451A(4)-451D(4) (e.g., square-shaped, rectangular-shaped, or polygonal-shaped lobe, or approximately square-shaped, approximately rectangular-shaped, or approximately polygonal-shaped lobe) connected via a connecting part 452A-452D to a central part 453. The central part 453 may be considered part of the electrodes 402A(4)-402D(4). Alternatively, in other embodiments, the central part 453 may be considered, or may include, a separate electrode from the electrodes 402A(4)-402D(4). The connecting parts 452A-452D may be considered part of the electrodes 402A(4)-402D(4) and/or the central part 453. The lobes 451A-451D, connecting parts 452A-452D, and the central part 453 may be made of the same material. In the embodiment depicted in FIG. 4E, the connecting parts 452A-452D may extend diagonally from the central part 453. For example, each connecting part 452 may extend from a corner of the central part 453 when the apparatus 400(4) includes four electrodes 402. Similar descriptions and Figure labelling notations apply to transducers 400(5) and 400(6) in FIGS. 4F and 4G, respectively, with differences as noted below.

In FIG. 4F, each electrode 402A(5)-402D(5) includes a lobe 451A′-451D′ (e.g., square-shaped, rectangular-shaped, or polygonal-shaped lobe, or approximately square-shaped, approximately rectangular-shaped, or approximately polygonal-shaped lobe) connected via a connecting part 452A′-452D′ to a central part 453′. The central part 453′ may be considered part of the electrodes 402A(5)-402D(5). Alternatively, the central part 453′ may be considered, or may include, a separate electrode from the electrodes 402A(5)-402D(5). The connecting parts 452A′-452D′ may be considered part of the electrodes 402A(5)-402D(5) and/or the central part 453′. The lobes 451A′-451D′, connecting parts 452A′-452D′, and the central part 453′ may be made of the same material. In the embodiment depicted in FIG. 4F, the connecting parts 452A′-452D′ may extend diagonally off-center from the central part 453′. For example, each connecting part 452′ may extend from a point adjacent to a corner of the central part 453′ when the apparatus 400 includes four electrodes 402. FIGS. 4E and 4F both illustrate at least four electrodes and C4 rotational symmetry around the centroid 484. However, the embodiment of FIG. 4E also has mirror plane symmetry through the centroid 484 (in both x and y directions)—the two sides of the 452A-452D connecting parts connect equidistant from the vertex of the central part of 453. The embodiment of FIG. 4F is lacking such mirror plane symmetry through the centroid 484.

FIG. 4G depicts an alternative embodiment of the four-lobed array of FIG. 4E, with different size and shape lobes, and wherein the connecting parts 453A″-453D″ are minimized in length such that the four lobes effectively merge with the central part 453″. In FIG. 4G, each electrode 402A(6)-402D(6) includes a lobe 451A″-451D″ (e.g., square-shaped, rectangular- shaped, or polygonal-shaped lobe, or approximately square-shaped, approximately rectangular- shaped, or approximately polygonal-shaped lobe) connected via a connecting part 452A″-452D″ to a central part 453″. The central part 453″ may be considered part of the electrodes 402A(6)- 402D(6). Alternatively, the central part 453″ may be considered, or may include, a separate electrode from the electrodes 402A(6)-402D(6). The connecting parts 452A″-452D″ may be considered part of the electrodes 402A(6)-402D(6) and/or the central part 453″. The lobes 451A″-451D″, connecting parts 452A″-452D″, and the central part 453″ may be made of the same material. In the embodiment depicted in FIG. 4G, the lobes 451A″-451D″ cover a larger surface area than the lobes 451A-451D and 451A′-451D′ depicted in FIGS. 4E and 4F, respectively.

In the embodiments of FIGS. 4E, 4F and 4G, the bandage overlay 480 is disposed over the array of electrodes 402A-402D such that the bandage overlay 480 covers the array of electrodes 402A-402D and void spaces 478A-478D between the electrodes 402A-402D. The transducer apparatus 400 may be capable of stretching such that a void space 478 located between at least one pair of adjacent electrodes 402 of the array is increased in size due to movement of one or more electrodes 402 of at least one pair of adjacent electrodes 402 of the array in a direction away from one another, for example, as shown in FIG. 4D. In some embodiments, the transducer apparatus 400 may be configured to stretch upon or after application of the apparatus on a subject. For each of the embodiments of FIGS. 4E, 4F and 4G, further embodiments exist wherein the bandage overlay 480 has at least one cut or slit 478 (FIG. 4B) extending from an outer edge of the bandage overlay 480 toward a center portion of the bandage overlay 480. The cuts or slits 478 formed in the bandage overlay 480 may be at least partially coincident with one or more of the void spaces 478A-478D between the electrodes 402A-402D. Transducer apparatus positioning and resulting flexibility with respect to the transducer apparatus and polyurethane film or bandage can be similar for the embodiments of FIGS. 4E, 4F and 4G.

The array of electrodes 402 (and elsewhere herein) may include electrodes of various sizes and shapes. In some embodiments, each electrode of an array of electrodes is of similar size and shape. Similarly, the area of the array of electrodes situated over the centroid 484 of an apparatus 400 may be of varying size.

For these arrays, and for any of the arrays disclosed herein, additional embodiments exist wherein a portion of one or more of the electrodes (or layer of anisotropic material, or layer of the transducer) is cut out or shaped with an indented concave surface on the periphery of the electrode (or layer of anisotropic material, or layer of the transducer) in order to accommodate a chemotherapy port or similar opening mechanism on the subject's body.

FIGS. 5A and 5B depict an example layout of an array of electrode elements 502A-502D disposed on the substrate layer 580 (e.g., polyurethane film or bandage) of a transducer apparatus 500 (500(1) in FIG. 5A and 500(2) in FIG. 5B), optionally paired with a layer of anisotropic material 572 (shown in FIG. 5B, but not FIG. 5A where it may either not be present or may be coincident with the areal trace of the electrodes). The bandage 580 may be composed of the same material as bandage overlay 480 (or 470) discussed above. In some embodiments, a front face of the array of electrodes 502A-502D faces a subject's body, and the layer of anisotropic material 572 covers the front face of the array of electrodes 502A-502D and extends (radially) outwardly from each electrode 502 to at least partially cover each void space 578A-578D in the array. In some embodiments, the layer of anisotropic material 572 may be composed of graphite (such as, for example, pyrolytic graphite). In further embodiments, bandage 580 covers the array of electrodes 502A-502D and the layer of anisotropic material 572 and extends (radially) outwardly from the combined areal footprint of each electrode 502 and associated layer of anisotropic material to at least partially cover each void space 578A-578D in the array (covering more than the areal footprint of the layer of anisotropic material 572). In some embodiments, the bandage 580 completely covers each void space 578A-578D.

In some embodiments, the apparatus 500 includes at least four electrodes 502. In some embodiments, the array of electrodes 502A-502D has point symmetry. Transducer apparatus 500 may include an array of electrode elements 502A-502D arranged around a centroid 584. For example, the array of electrodes may include four electrodes having point symmetry (C4 symmetry) about a centroid 584. Each electrode may be substantially similar in size and shape. In some embodiments, the bandage 580 may cover all of the electrodes and all of the void spaces between the electrodes. In some embodiments, the bandage 580 paired to the apparatus 500 includes one or more cutouts coincident with at least a portion of the void spaces 578A-578D between at least one of the pairs of electrodes 502. The cutouts may have an open shape so that the one or more cutouts define one or more concave portions along an outer edge of the bandage 580 when viewed from a direction perpendicular to the face of the array (FIGS. 5A and 5B). For the embodiments of FIGS. 5A and 5B, a 45° rotation of the existing electrode positions about the centroid 584 positions each void space over the former existing electrode position, thereby providing relief to the areas of skin that may have experienced skin irritation from the electrodes. Furthermore, the bandage (e.g., polyurethane film or bandage) provides flexibility to the transducer array apparatus and allows the array to accommodate movements of the skin due to movements of the torso of the subject. Although shown for 4 electrodes and arrays with C4 rotational symmetry, similar constructs with other rotational symmetry are readily envisioned (e.g., with 5, 6, or more electrodes), as well as other electrode arrays spaced and arranged to allow for a translational shift of the electrode array.

FIG. 5C illustrates a similar transducer array 500(3) as that in FIG. 5B (with similar labelling and description applicable) except instead of having open cut-outs that define concave portions along an outer edge of the bandage 580, the FIG. 5C embodiment illustrates closed cut-outs that are coincident with the void spaces 578 that may be rotated to position each void space over each former existing electrode position. The cut-outs may be made in just the layer of anisotropic material 572 (and any associated conductive adhesive layer(s)), or may be made through the layer of anisotropic material and the bandage. In an alternative embodiment, an additional substrate layer may be positioned between the electrodes 502 (optionally with the layer of anisotropic material) and the bandage overlay 580, and the closed cut-outs may go through the layer of anisotropic material in areas coincident with at least a portion of one or more (or each of the) void spaces between the electrodes leaving this additional substrate layer exposed in the void space areas. Preferably this area is free of adhesive. Preferably this additional substrate layer has some flexibility (for example, it may be a non-woven, cloth or gauze material). In this embodiment, the cut out region may present just the substrate in the void space, or a region of medication may be introduced onto or into this exposed area of the substrate. In the latter scenario (with medication), rotation of the void space onto areas of former existing electrode positions may bring medicated relief to the areas of skin that may have experienced skin irritation from the electrodes. Transducer array apparatus positioning and resulting flexibility with respect to the transducer array apparatus and polyurethane film or bandage can be similar for the embodiments of FIGS. 5A, 5B and 5C. The embodiments of FIGS. 5B and 5C can provide similar resulting utility both in terms of providing relief to areas of skin irritation, and in terms of transducer array flexibility, although the embodiment of FIG. 5C can additionally provide relief with or without medication to areas of skin irritation.

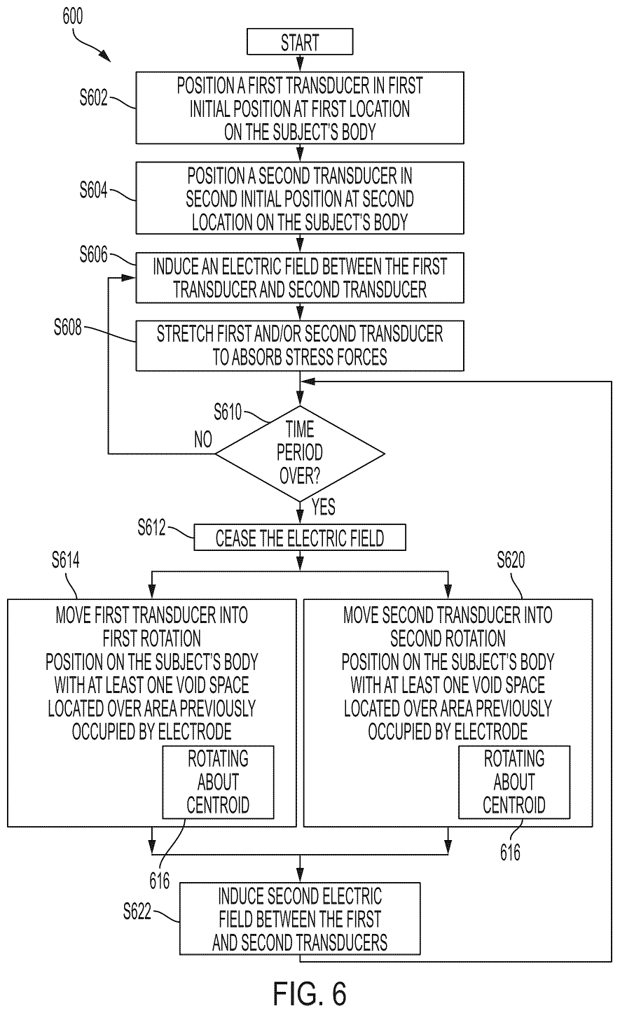

FIG. 6 depicts an example method 600 of applying TTFields to a subject's body in accordance with the present techniques. The method 600 begins at step S602 with positioning a first transducer in a first initial position at a first location of the subject's body. The first transducer may include a plurality of electrodes, a substrate layer, and/or a bandage layer as described above. In certain embodiments, the first transducer may include a plurality of void spaces located between adjacent electrodes (e.g., as shown in the apparatuses of FIGS. 4C-5C). Optionally, the transducer array may comprise a layer of anisotropic material as described herein.

At step S604, the method 600 may include positioning a second transducer in a second initial position at a second location of the subject's body. The second transducer may include a plurality of electrodes in initial electrode positions, a substrate layer, and/or a bandage layer as described above. In certain embodiments, the second transducer may include a plurality of void spaces located between adjacent electrodes (e.g., as shown in the apparatuses of FIGS. 4C-5C). Optionally, the transducer array may comprise a layer of anisotropic material as described herein.

At step S606, the method 600 includes inducing an electric field between the first transducer located in a first initial position at the first location of the subject's body and the second transducer in a second initial position located at the second location of the subject's body.

At step S608, the method 600 includes stretching the first transducer and/or the second transducer to absorb one or more stress force due to movements of the subject's body. For example, stretching of the transducer may occur such that a void space located between at least one pair of adjacent electrodes of the array is increased in size due to movement of one or more electrodes of at least one pair of adjacent electrodes of the array in a direction away from one another, for example as shown in FIG. 4D.

At step S610, the method 600 includes determining whether a first period of time has passed. After inducing the electric field for more than the first period of time, the method 600 proceeds to step S612, which includes ceasing the electric field.

At step S614, the method 600 includes moving the first transducer into a first rotation position on the subject's body at the first location. In an example, at step S614 moving the first transducer to the first rotation position may include rotating (616) the first transducer about its centroid. In particular, moving the first transducer may include rotating the first transducer about its centroid into a first rotation position at the first location of the subject's body. In some embodiments, in the first rotation position, all areas that were not previously covered by an electrode in the first initial position may now be covered by an electrode, and vice-versa.

The method 600 may also include, at step S620, moving the second transducer from a second initial position at a second location on the subject's body into a second rotation position on the subject's body (in analogous fashion to that described above for the first transducer in step 614). In some embodiments, in the second rotation position, all areas that were not previously covered by an electrode in the second initial position may now be covered by an electrode, and vice-versa. In an example, at step S620 moving the second transducer to the second rotation position may include rotating (616) the second transducer about its centroid (as described above for movement of the first transducer).

At step S622, the method 600 includes inducing another electric field between the first transducer and the second transducer.

While an order of operations is indicated in FIG. 6 for illustrative purposes, the timing and ordering of such operations may vary where appropriate without negating the purpose and advantages of the examples set forth in detail throughout the remainder of this disclosure.

### ILLUSTRATIVE EMBODIMENTS

The invention includes other illustrative embodiments (“Embodiments”) as follows.

Embodiment 1. A transducer apparatus for delivering tumor treating fields to a subject's body, the transducer apparatus comprising an array of electrodes, the array configured to be positioned over the subject's body with a face of the array facing the subject's body, the array comprising electrode elements positioned in substantially symmetrical positions arranged around a centroid of the array; a void space located between at least one pair of adjacent electrodes of the array; and a polymeric material layer overlaying the array of electrodes and located on a side of the array facing away from the subject's body.

Embodiment 1A: The apparatus of Embodiment 1, wherein the polymeric material layer is flexible and stretchable.

Embodiment 2: The apparatus of Embodiment 1, wherein the polymeric material layer comprises polyurethane.

Embodiment 3: The apparatus of Embodiment 2, wherein the polymeric material layer comprises a polyurethane polymer film or bandage.

Embodiment 4: The apparatus of Embodiment 1, wherein the polymeric material layer has a thickness of less than or equal to 250 μm.

Embodiment 4A: The apparatus of Embodiment 1, wherein the polymeric material layer has a thickness of less than or equal to 200 μm, or less than or equal to 160 μm, such as, for example, in a range of from 50-250 μm, or from 50-200 μm, or from 50-160 μm.

Embodiment 4B: The apparatus of Embodiment 4, the polymeric material layer has a thickness in a range of from 80-160 μm, or from 100-140 μm.

Embodiment 4C: The apparatus of Embodiment 1, wherein the array of electrodes includes at least four electrodes.

Embodiment 5: The apparatus of Embodiment 1, wherein the array of electrodes has point symmetry (rotational symmetry).

Embodiment 5A: The apparatus of Embodiment 1, wherein the array of electrodes includes four electrodes having point symmetry about the centroid.

Embodiment 5B: The apparatus of Embodiment 1, wherein each electrode is substantially similar in size and shape.

Embodiment 6: The apparatus of Embodiment 1, wherein the polymeric material layer is disposed over the array of electrodes such that the polymeric material layer covers the array of electrodes and the void space in the array.

Embodiment 7: The apparatus of Embodiment 1, the polymeric material layer substantially covers the array of electrodes, and the polymeric material layer has one or more cutouts formed therein, at least one of the one or more cutouts being coincident with at least a portion of the void space located between at least one pair of adjacent electrodes in the array.

Embodiment 8: The apparatus of Embodiment 7, wherein the one or more cutouts have an open shape so that the one or more cutouts define one or more concave portions along an outer edge of the polymeric material layer when viewed from a direction perpendicular to the face of the array.

Embodiment 8A: The apparatus of Embodiment 7, wherein the one or more cutouts have a closed shape so that the one or more cutouts are surrounded by the polymeric material layer when viewed from a direction perpendicular to the face of the array.

Embodiment 9: The apparatus of Embodiment 1, wherein the apparatus is capable of stretching such that the void space located between at least one pair of adjacent electrodes of the array is increased in size due to movement of one or more electrodes of at least one pair of adjacent electrodes of the array in a direction away from one another.

Embodiment 9A: The apparatus of Embodiment 9, wherein the apparatus is configured to stretch upon or after application of the apparatus on a subject.

Embodiment 10: A transducer apparatus for delivering tumor treating fields to a subject's body, the transducer apparatus comprising an array of electrodes, the array configured to be positioned over the subject's body with a face of the array facing the subject's body; a plurality of void spaces, each void space located between at least one pair of adjacent electrodes of the array; a layer of anisotropic material electrically coupled to the array of electrodes and located on a side of the array facing the subject's body; and a polymeric material layer overlaying the array of electrodes and the layer of anisotropic material and located on a side of the array facing away from the subject's body.

Embodiment 10A: The apparatus of Embodiment 10, wherein the polymeric material layer is flexible and stretchable.

Embodiment 11: The transducer apparatus of Embodiment 10, wherein the layer of anisotropic material comprises graphite.

Embodiment 11A: The transducer apparatus of Embodiment 10, wherein the layer of anisotropic material comprises pyrolytic graphite, graphitized polymer, or graphite foil made from compressed high purity exfoliated mineral graphite.

Embodiment 11B: The transducer apparatus of Embodiment 10, wherein the layer of anisotropic material has a front face and a back face, wherein the back face of the layer of anisotropic material faces the array of electrodes, wherein the layer of anisotropic material has different thermal and/or electrical conductivities in a direction perpendicular to the front face than in directions that are parallel to the front face.

Embodiment 11C: The transducer apparatus of Embodiment 11B, wherein the layer of anisotropic material has a first electrical resistance in a direction that is perpendicular to the front face, and resistance of the sheet in directions that are parallel to the front face is less than half of the first resistance.

Embodiment 11D: The transducer apparatus of Embodiment 11B, wherein the layer of anisotropic material has a first electrical resistance in a direction that is perpendicular to the front face, and resistance of the sheet in directions that are parallel to the front face is less than 10%, or less than 1%, or less than 0.1% of the first resistance.

Embodiment 11E: The transducer apparatus of Embodiment 11B, wherein the layer of anisotropic material has a first thermal conductivity in a direction that is perpendicular to the front face, and thermal conductivity of the sheet in directions that are parallel to the front face is more than two times higher than the first thermal conductivity.

Embodiment 11F: The transducer apparatus of Embodiment 11B, wherein the layer of anisotropic material has a first thermal conductivity in a direction that is perpendicular to the front face, and thermal conductivity of the sheet in directions that are parallel to the front face is more than 10 times, or more than 100 times, or more than 1,000 times higher than the first thermal conductivity.

Embodiment 12: The transducer apparatus of Embodiment 10, wherein a front face of the array of electrodes faces the subject's body, and wherein the layer of anisotropic material is positioned over the front face of the array of electrodes, including over each electrode of the array of electrodes, and extends outwardly from each electrode to at least partially cover each void space in the array.

Embodiment 13: The transducer apparatus of Embodiment 10, wherein the polymeric material layer comprises polyurethane.

Embodiment 14: The transducer apparatus of Embodiment 13, wherein the polymeric material layer comprises a polyurethane polymer film or bandage.

Embodiment 14A: The apparatus of Embodiment 13 or 14, wherein the polymeric material layer has a thickness of less than or equal to 250 μm.

Embodiment 14B: The apparatus of Embodiment 13 or 14, wherein the polymeric material layer has a thickness of less than or equal to 200 μm, or less than or equal to 160 μm, such as, for example, in a range of from 50-250 μm, or from 50-200 μm, or from 50-160 μm.

Embodiment 14C: The apparatus of Embodiment 13 or 14, wherein the polymeric material layer has a thickness in a range of from 80-160 μm, or from 100-140 μm.

Embodiment 15: The transducer apparatus of Embodiment 10, wherein the polymeric material layer covers the layer of anisotropic material and extends outwardly from the layer of anisotropic material to at least partially cover each void space in the array.

Embodiment 16: The transducer apparatus of Embodiment 10, wherein a front face of the array of electrodes faces the subject's body, and wherein the layer of anisotropic material is positioned over the front face of the array of electrodes, including over each electrode of the array of electrodes, and at least partially covers each void space in the array, wherein the polymeric material layer covers the layer of anisotropic material and at least partially covers each void space in the array, and wherein the polymeric material layer covers more of each void space in the array than the layer of anisotropic material.

Embodiment 16A: The transducer apparatus of Embodiment 16, wherein the polymeric material layer substantially covers the layer of anisotropic material, and the polymeric material layer has one or more cutouts formed therein, at least one of the one or more cutouts being coincident with at least a portion of the void space in the array.

Embodiment 16B: The transducer apparatus of Embodiment 16A, wherein the one or more cutouts have an open shape so that the one or more cutouts define one or more concave portions along an outer edge of the polymeric material layer when viewed from a direction perpendicular to the face of the array.

Embodiment 16C: The transducer apparatus of Embodiment 16A, wherein the one or more cutouts have a closed shape so that the one or more cutouts are surrounded by the polymeric material layer when viewed from a direction perpendicular to the face of the array.

Embodiment 16D: The transducer apparatus of claim Embodiment 16C, wherein at least a portion of each of the one or more cutouts in the polymeric material layer is coincident with at least a portion of a cutout in the layer of anisotropic material.

Embodiment 16E: The transducer apparatus of claim Embodiment 16C, wherein each of the one or more cutouts in the polymeric material layer is coincident with a cutout in the layer of anisotropic material.

Embodiment 17: The transducer apparatus of Embodiment 16, wherein the polymeric material layer covers each void space in the array.

Embodiment 18: The transducer apparatus of Embodiment 10, further comprising at least one of: conductive adhesive material located on a front face of the layer of anisotropic material opposite the array of electrodes, or conductive adhesive material located between the array of electrodes and a back face of the layer of anisotropic material facing the array of electrodes.

Embodiment 19: The transducer apparatus of Embodiment 10, wherein the array of electrodes includes four electrodes, or at least four electrodes, having point symmetry about the centroid.

Embodiment 20: The transducer apparatus of Embodiment 10, wherein the apparatus is capable of stretching such that the void space located between at least one pair of adjacent electrodes of the array is increased in size due to movement of one or more electrodes of at least one pair of adjacent electrodes of the array in a direction away from one another.

Embodiment 20A: The apparatus of Embodiment 20, wherein the apparatus is configured to stretch upon or after application of the apparatus on a subject.

Embodiment 20B: A transducer apparatus for delivering tumor treating fields to a subject's body, the transducer apparatus comprising: an array of electrodes, the array configured to be positioned over the subject's body with a face of the array facing the subject's body; a layer of anisotropic material electrically coupled to the array of electrodes and located on a side of the array facing the subject's body and having a plurality of slits or cuts in the layer of anisotropic material, each of the slits or cuts located between a pair of adjacent electrodes of the array; and a polymeric material layer overlaying the array of electrodes and the layer of anisotropic material and located on a side of the array facing away from the subject's body.

Embodiment 20C: The transducer apparatus of Embodiment 20B, wherein the polymeric material layer has one or more slits or cuts coincident with one or more of the plurality of slits or cuts in the layer of anisotropic material.

Embodiment 21: The transducer apparatus of Embodiment 10, wherein the array comprises electrode elements positioned in existing electrode positions arranged around a centroid of the array.

Embodiment 22: The transducer apparatus of Embodiment 21, wherein at least one void space in the array is capable of enclosing an areal footprint equivalent to at least a portion of an areal footprint of at least one existing electrode position, and is superimposable on at least a portion of at least one existing electrode position by rotation of the array around the centroid.

Embodiment 23: The transducer apparatus of Embodiment 21, wherein at least one void space in the array is capable of enclosing an areal footprint equivalent to at least half of an areal footprint of at least one existing electrode position, and is superimposable on at least half of at least one existing electrode position by rotation of the array around the centroid.

Embodiment 24: The transducer apparatus of Embodiment 21, wherein at least one void space in the array is capable of enclosing an areal footprint equivalent to at least an areal footprint of at least one existing electrode position, and is superimposable on at least one existing electrode position by rotation of the array around the centroid.

Embodiment 25: A method of applying tumor treating fields to a subject's body, the method comprising locating a first transducer apparatus in a first position at a first location on the subject's body, the first transducer apparatus comprising an array of electrodes, the array configured to be positioned over the subject's body with a face of the array facing the subject's body, the array comprising electrodes positioned in existing electrode positions arranged around a centroid of the array; a void space located between at least one pair of adjacent electrodes of the array; and a polymeric material layer overlaying the array of electrodes and located on a side of the array facing away from the subject's body; stretching the transducer apparatus either upon or after application of the apparatus to the subject's body in order to absorb one or more stress force due to movements of the subject's body while substantially maintaining the transducer in the first position; and inducing an electric field between the first transducer and a second transducer located at a second location on the subject's body.

Embodiment 26: The method of Embodiment 25, wherein when the transducer is stretched, at least one void space located between at least one pair of adjacent electrodes of the array is increased in size due to movement of one or more electrodes of at least one pair of adjacent electrodes of the array in a direction away from one another and away from at least one existing electrode position.

Optionally, for each embodiment described herein, the voltage generation components supply the transducers with an electrical signal having an alternating current waveform at frequencies in a range from about 50 kHz to about 1 MHz and appropriate to deliver TTFields treatment to the subject's body. In some embodiments, the electrical signal has an alternating current waveform at frequencies in a range from about 100 kHz to about 500 kHz and appropriate to deliver TTFields treatment to the subject's body.

Embodiments illustrated under any heading or in any portion of the disclosure may be combined with embodiments illustrated under the same or any other heading or other portion of the disclosure unless otherwise indicated herein or otherwise clearly contradicted by context. For example, and without limitation, embodiments described in dependent claim format for a given embodiment (e.g., the given embodiment described in independent claim format) may be combined with other embodiments (described in independent claim format or dependent claim format).

Numerous modifications, alterations, and changes to the described embodiments are possible without departing from the scope of the present invention defined in the claims. It is intended that the present invention not be limited to the described embodiments, but that it has the full scope defined by the language of the following claims, and equivalents thereof.

What is claimed is:

1. A transducer apparatus for delivering tumor treating fields to a subject's body, the transducer apparatus comprising:
an array of electrodes, the array configured to be positioned over the subject's body with a face of the array facing the subject's body, the array comprising electrode elements positioned in substantially symmetrical positions arranged around a centroid of the array;
a void space located between at least one pair of adjacent electrodes of the array; and
a polymeric material layer overlaying the array of electrodes and located on a side of the array facing away from the subject's body.

2. The transducer apparatus of claim 1, wherein the polymeric material layer comprises polyurethane.

3. The transducer apparatus of claim 2, wherein the polymeric material layer comprises a polyurethane polymer film or bandage.

4. The transducer apparatus of claim 1, wherein the polymeric material layer has a thickness of less than or equal to 250 μm.

5. The transducer apparatus of claim 1, wherein the array of electrodes has point symmetry.

6. The transducer apparatus of claim 1, wherein the polymeric material layer is disposed over the array of electrodes such that the polymeric material layer covers the array of electrodes and the void space in the array.

7. The transducer apparatus of claim 1, wherein:
the polymeric material layer substantially covers the array of electrodes, and
the polymeric material layer has one or more cutouts formed therein, at least one of the one or more cutouts being coincident with at least a portion of the void space located between at least one pair of adjacent electrodes in the array.

8. The transducer apparatus of claim 7, wherein the one or more cutouts have an open shape so that the one or more cutouts define one or more concave portions along an outer edge of the polymeric material layer when viewed from a direction perpendicular to the face of the array.

9. The transducer apparatus of claim 1, wherein the apparatus is capable of stretching such that the void space located between at least one pair of adjacent electrodes of the array is increased in size due to movement of one or more electrodes of at least one pair of adjacent electrodes of the array in a direction away from one another.

10. A transducer apparatus for delivering tumor treating fields to a subject's body, the transducer apparatus comprising:
an array of electrodes, the array configured to be positioned over the subject's body with a face of the array facing the subject's body;
a plurality of void spaces, each void space located between at least one pair of adjacent electrodes of the array;
a layer of anisotropic material electrically coupled to the array of electrodes and located on a side of the array facing the subject's body; and
a polymeric material layer overlaying the array of electrodes and the layer of anisotropic material and located on a side of the array facing away from the subject's body.

11. The transducer apparatus of claim 10, wherein the layer of anisotropic material comprises graphite.

12. The transducer apparatus of claim 10, wherein a front face of the array of electrodes faces the subject's body, and wherein the layer of anisotropic material is positioned over the front face of the array of electrodes, including over each electrode of the array of electrodes, and extends outwardly from each electrode to at least partially cover each void space in the array.

13. The transducer apparatus of claim 10, wherein the polymeric material layer comprises polyurethane.

14. The transducer apparatus of claim 13, wherein the polymeric material layer comprises a polyurethane polymer film or bandage.

15. The transducer apparatus of claim 10, wherein the polymeric material layer covers the layer of anisotropic material and extends outwardly from the layer of anisotropic material to at least partially cover each void space in the array.

16. The transducer apparatus of claim 10, wherein a front face of the array of electrodes faces the subject's body, and wherein the layer of anisotropic material is positioned over the front face of the array of electrodes, including over each electrode of the array of electrodes, and at least partially covers each void space in the array,
wherein the polymeric material layer covers the layer of anisotropic material and at least partially covers each void space in the array, and
wherein the polymeric material layer covers more of each void space in the array than the layer of anisotropic material.

17. The transducer apparatus of claim 16, wherein the polymeric material layer covers each void space in the array.

18. The transducer apparatus of claim 10, further comprising at least one of:
conductive adhesive material located on a front face of the layer of anisotropic material opposite the array of electrodes, or conductive adhesive material located between the array of electrodes and a back face of the layer of anisotropic material facing the array of electrodes.

19. The transducer apparatus of claim 10, wherein the array of electrodes includes at least four electrodes having point symmetry about the centroid.

20. The transducer apparatus of claim 10, wherein the apparatus is capable of stretching such that the void space located between at least one pair of adjacent electrodes of the array is increased in size due to movement of one or more electrodes of at least one pair of adjacent electrodes of the array in a direction away from one another.

21. The transducer apparatus of claim 16, wherein:
the polymeric material layer substantially covers the layer of anisotropic material, and the polymeric material layer has one or more cutouts formed therein, at least one of the one or more cutouts being coincident with at least a portion of the void space in the array.

22. The transducer apparatus of claim 21, wherein the one or more cutouts have an open shape so that the one or more cutouts define one or more concave portions along an outer edge of the polymeric material layer when viewed from a direction perpendicular to the face of the array.

23. The transducer apparatus of claim 21, wherein the one or more cutouts have a closed shape so that the one or more cutouts are surrounded by the polymeric material layer when viewed from a direction perpendicular to the face of the array.

24. The transducer apparatus of claim 23, wherein at least a portion of each of the one or more cutouts in the polymeric material layer is coincident with at least a portion of a cutout in the layer of anisotropic material.

25. A transducer apparatus for delivering tumor treating fields to a subject's body, the transducer apparatus comprising:
an array of electrodes, the array configured to be positioned over the subject's body with a face of the array facing the subject's body;
a layer of anisotropic material electrically coupled to the array of electrodes and located on a side of the array facing the subject's body and having a plurality of slits or cuts in the layer of anisotropic material, each of the slits or cuts located between a pair of adjacent electrodes of the array; and
a polymeric material layer overlaying the array of electrodes and the layer of anisotropic material and located on a side of the array facing away from the subject's body.

26. The transducer apparatus of claim 25, wherein the polymeric material layer has one or more slits or cuts coincident with one or more of the plurality of slits or cuts in the layer of anisotropic material.
